(19) United States
(12) Patent Application Publication
(10) Pub. No.: US 2026/0077123 A1
(43) Pub. Date: Mar. 19, 2026

(54) NEEDLE AND ASSOCIATED METHOD OF MANUFACTURE

(71) Applicant: Unomedical A/S, Lejre (DK)

(72) Inventors: Erik Othel-Jacobsen, Snekkersten (DK); Henrik Tipsmark, Roskilde (DK); Rune Frimand Byager, Hvalsoe (DK); Jesper Erichsen, Roskilde (DK)

(21) Appl. No.: 19/393,953

(22) Filed: Nov. 19, 2025

Related U.S. Application Data
(63) Continuation of application No. PCT/EP2024/063783, filed on May 17, 2024.
(60) Provisional application No. 63/503,247, filed on May 19, 2023.
(60) Provisional application No. 63/503,290, filed on May 19, 2023.

(30) Foreign Application Priority Data
Jul. 6, 2023 (GB) 2310426.8
Jul. 6, 2023 (GB) 2310435.9

(51) Int. Cl. A61M 5/158; A61L 29/02; B21G 1/00

(52) U.S. Cl. CPC A61M 5/158; A61L 29/02; B21G 1/003; A61L 2400/16; A61M 2005/1585; A61M 2205/0216; A61M 2207/00

(57) ABSTRACT

There is provided a needle comprising a needle tube extending along a central axis between proximal and distal ends of the needle, the needle tube defining a lumen, and the distal end defining an opening into the lumen. The needle comprises a point, a cutting edge, and a heel extending between inner and outer surfaces of the needle tube. At least a region of the heel extends substantially orthogonally to the central axis to provide a non-cutting edge of the distal end.

### TECHNICAL FIELD

The invention relates generally to a needle for percutaneous use. In particular, the invention relates to a needle tip geometry of such a needle configured to avoid coring, and also to a method of manufacturing such a needle. The invention also relates to a flexible needle for use in an infusion device. In certain embodiments, the needle may be formed of a nickel titanium alloy, i.e. nitinol.

### BACKGROUND

Needles are a typical feature of a wide variety of medical devices, and are commonly used in many percutaneous medical procedures, including injection, infusion and blood sampling. Insertion of a needle into soft tissue is a process involving cutting of the tissue, and is dependent upon the cutting edge geometry of the tip of the needle. The geometry is designed to cut through the tissue effectively to minimise insertion forces, and thereby minimise tissue deformation and needle deflection during insertion. It is generally recognised that minimising tissue deformation eases pain and tissue trauma associated with insertion, and minimising needle deflection improves placement of the needle.

Needles are available in a wide variety of configurations, and commonly include at least one bevel. A bevel is an angled surface formed at the tip of the needle when it is sharpened to make a needle point. Some needles include multiple bevels, for example a lancet needle—the most common needle used in medical applications—has three bevelled surfaces: the primary bevel, which is typically formed by grinding the tip at an angle, and two secondary bevels, which are typically formed by grinding opposite sides of the primary bevel to produce the needle point and a cutting edge. Diametrically opposite the needle point is the heel of the needle, which is sometimes referred to as the heel of the bevel.

During insertion, an arcuate cut is made in the tissue, extending from each side of the needle point as the needle advances. As the cut continues to extend, a hinged flap of tissue overlaying the lumen of the needle is formed. The heel may provide a cutting edge that further extends the cut such that the flap is severed, and subsequently the flap may be pushed into the lumen by further advancement of the needle. This undesirable mechanism is known as coring. Coring may similarly occur when a needle is inserted through a pharmaceutical closure, i.e. a bung or plug, of a container of medicament to draw up medicament therefrom, wherein a fragment of the material forming the closure is cut away. Coring is undesirable as it reduces the efficacy of the needle, and consequently the efficacy of delivery of medicament to a patient.

Coring may lead to contamination of medicament and/or injection of a foreign body, for example a severed fragment of the material forming pharmaceutical closure, into a patient. Coring may increase foreign body response (FBR) to the medical procedure in question. Coring has led to the development of non-coring needles, wherein the heel is sandblasted and thereby dulled to ensure the heel provides a non-cutting edge. This is an additional process step, and may be difficult or undesirable when processing certain materials used to manufacture needles. In particular, sandblasting may be difficult when manufacturing needles formed of a superelastic alloy, for example a nickel titanium alloy, i.e. nitinol, due to its high flexibility.

Devices like subcutaneous infusion sets for insulin or other medications use a small rigid needle to insert a soft cannula under the skin. The needle, often referred to as the insertion needle is then removed, leaving the cannula in place. Insertion needles can add to the pain and tissue trauma associated with insertion due to size differences between the needle and the cannula. More particularly, by having a soft cannula around the needle, the wound created by the needle may be too small for the soft cannula. The cannula therefore typically needs to be pressed through the smaller hole, which will cause more pain to the patient. Moreover, many soft cannulas do not sufficiently resist kinking, which can stop the flow of the therapeutic agent. On the other hand, if the infusion are performed with a conventional needle, the tip of the rigid needle will engage the adjacent tissue during patient movement, which may worsen the wound and cause additional trauma to the patient. For these reasons among others, there remains a need for further improvements in this technological field.

It would be useful to provide a needle inserter assembly, as well as a method of inserting a needle into the soft tissue of a patent, to reliably insert a needle at an optimal angle to the soft tissue, that is the insertion axis is reliably controlled. In particular, it would be useful to enable a needle to be inserted in a way that reduces or minimises lateral forces on the needle as it is inserted. In other words, it would be useful to insert a needle so that the urging force applied to the needle to insert the needle into the soft tissue acts along the needle insertion axis.

It would also be useful to simplify the operation of needle inserter assembly. It would be useful to provide convenient insertion of a needle into soft tissue, particularly so that the needle is securely mounted to the soft tissue for use over a prolonged period, such as several days or more. It would be useful to provide a flexible needle that is insertable into soft tissue that provides a reliable flow through the lumen of the needle.

It is an object of embodiments of the invention to provide a needle that at least mitigates one or more problems associated with known arrangements. The invention is defined by the appended claims.

### SUMMARY OF THE DISCLOSURE

According to an aspect of the disclosure, there is provided a needle comprising a needle tube extending along a central axis between proximal and distal ends of the needle, the needle tube defining a lumen, and the distal end defining an opening into the lumen and comprising: a point, a cutting edge, and a heel extending between inner and outer surfaces of the needle tube, at least a region of the heel extending substantially orthogonally to the central axis to provide a non-cutting edge of the distal end. This arrangement is intended to provide a non-coring needle. In certain embodiments, the purpose of the point may be to facilitate insertion of the needle, for example into patient tissue, and/or the purpose of the cutting edge may be to make a cut during insertion of the needle, as the needle advances, for example in the patient tissue.

In certain embodiments, the substantially orthogonal region may be a first substantially planar surface (i.e. a first surface, the first surface being planar) extending over a radial angle measured along an inner surface of the needle tube. The radial angle may be at least approximately 30° and/or up to approximately 180°. Additionally, or alternatively, the cutting edge may comprise a second surface extending from at least one side of the needle tip. The cutting edge may comprise a third surface, and each of the second and third surfaces extend from a respective side of the needle tip. The second and third surfaces may extend symmetrically to one another on their respective side of the needle tip, and/or one or both of the second and third surfaces may be a substantially planar surface. Additionally, or alternatively, the point may be diametrically opposite the centre of the heel.

In certain embodiments, the heel may extend about the needle tube to form a cut-out or slot therein extending substantially axially from the opening, i.e. the heel may comprise a cut-out or slot extending substantially axially along the needle tube from the opening. The cut-out may have a width equal to the inner diameter of the needle tube. Additionally, or alternatively, the cut-out may be delimited by opposing fourth and fifth substantially planar surfaces (i.e. a fourth surface and a fifth surface, the fourth fifth surfaces being planar) extending between the inner and outer surfaces of the needle tube. At least one of the fourth and fifth substantially planar surfaces may extend tangentially from the inner surface of the needle tube.

In certain embodiments, the needle may be formed of a superelastic material, for example a nickel titanium alloy, i.e. nitinol.

According to another aspect of the disclosure, there is provided a method of manufacturing a needle, the method comprising: cutting a needle tube to form an end thereof comprising a point, a cutting edge and a heel, wherein cutting the needle tube to form the heel includes cutting a region of the heel to extend between inner and outer surfaces of the needle tube substantially orthogonal to a central axis of the needle tube. Cutting may be laser cutting, for example thermal laser cutting and/or femtosecond laser cutting. Cutting the needle tube include cutting the needle tube to form any of the features of, or relating to, the needle of above-described aspect.

According to another aspect of the disclosure, there is provided a needle having a lumen for distributing fluid into, or removing fluid from, a soft tissue of a patient, the needle includes: a proximal portion, including a first port of the lumen, and a distal portion, including a second port of the lumen: wherein the distal portion includes a sharpened tip provided on a first lengthwise side of the needle, and a deformable portion provided on an opposing, second lengthwise side of the needle, wherein the tip is configured to incise an entry point in the soft tissue of a patient as the tip is urged against the soft tissue by an urging force provided in a direction along the needle, and wherein the deformable portion is configured so that, in response to the urging force, the tip is deflectable away from the second lengthwise side of the needle as the tip is inserted into the soft tissue beyond the entry point. In this way, a flexible needle is provided that controls the degree of deflection as it is inserted into soft tissue of a patient. The needle is thereby inserted along an insertion axis at a predetermined angle or at a predetermined range of angles, without risk of kinking or bending, or otherwise impeding fluid flow in the lumen of the needle. The needle may be adapted to conveniently provide an optimum insertion angle.

In certain embodiments, the tip may be deflectable in response to an urging force in a range of from 1 N to 5 N, preferably from 2 N to 4 N, more preferably from 2.5 N to 3 N.

In certain embodiments, the deformable portion may include at least one aperture extending through the needle from an outer surface of the distal portion to the lumen. In this way, the deformable portion may be configured so that apertures are openable as the tip is deflected. The deformable portion may be configured to deform, in response to the urging force, from a compressed condition, in which apertures have a minimal open area, to an expanded condition, in which the apertures have a maximal open area.

In certain embodiments, the at least one aperture may be an elongate slot oriented to extend partially around a circumference of the distal portion. Each elongate slot may extend around the circumference of the distal portion in a range of from 30° to 180°, preferably in a range of from 45° to 150°, more preferably in a range of from 60° to 120°.

In certain embodiments, the at least one aperture may be a plurality of apertures spaced apart along the lumen in the lengthwise direction of the needle, typically with a spacing in the range 0.5 mm to 2 mm. That is, each aperture of the plurality of apertures may have a spacing from a neighbouring aperture in the range 0.5 mm to 2 mm.

In certain embodiments, the deformable portion may extend along the distal portion for a distance in a range of from 2 mm to 20 mm, preferably in a range of from 3 mm to 10 mm.

That is, the deformable portion may extend in the lengthwise direction along the distal portion for a distance in the range of from 2 mm to 20 mm, preferably in a range of from 3 mm to 10 mm.

In certain embodiments, the deformable portion may be configured so that in response to the urging force the distal portion is deformed up to 6%. For example, the deformable portion may be formed of an elastically deformable material, such as a super-elastic material or a pseudo-elastic material. Additionally, or alternatively, the deformable portion may be configured so that, in response to the urging force, the tip is deflected relative to the direction of the urging force at an angle in the range of from 20° to 40°, preferably 30°. At least in the distal portion, the lumen may include a circular cross-section extending within the needle, and where the first lengthwise side of the needle and the second lengthwise side of the needle are provided on opposing sides of the cross-section.

In certain embodiments, the needle may be elastically or super-elastically deformable. Accordingly, the needle may be formed from a pseudo-elastic material or a super-elastic material. The needle may be elastically deformable in a range of from greater than 0% to 9%, preferably a range of from greater than 0% to 6%. The needle may be formed from a nitinol material, typically a nitinol material with an austenite finish temperature in the range of from −15° C. to 30° C., preferably in the range of from 0° C. to 20° C.

In certain embodiments, the needle may have a wall thickness of defined between an outer surface and a lumen surface, and wherein the wall thickness is at least 20 μm. At least a portion of the needle may have a wall thickness of defined between a portion of the outer surface and a portion of the lumen surface, wherein the wall thickness is at least 20 μm. One or more of the distal portion, the intermediate portion, or the proximal portion of the needle may have a wall thickness of defined between respective portions of an outer surface and a lumen surface, wherein the wall thickness is at least 20 μm. More suitably, a wall thickness may be less than 400 μm.

In certain embodiments, the needle may comprise an outer diameter, wherein the outer diameter is less than 0.7 mm. At least a portion of the needle may have an outer diameter of less than 0.7 mm. One or more of the distal portion, the intermediate portion, or the proximal portion of the needle may have an outer diameter of less than 0.7 mm. An outer diameter may be at least 0.4 mm. The needle may comprise an inner diameter, wherein the inner diameter is at least 0.2 mm. At least a portion of the needle may have an inner diameter of at least 0.2 mm. One or more of the distal portion, the intermediate portion, or the proximal portion of the needle may have an inner diameter of at least 0.2 mm. An inner diameter may be less than 0.5 mm.

In certain embodiments, the deformable portion may be deflectable around an arcuate path with a pitch radius of at least 3.5 mm. Suitably, the pitch radius may be in a range for from 3.5 mm to 20 mm, preferably in a range of from 4 mm to 12 mm.

According to another aspect of the disclosure, there is provided a needle insertion assembly including a support member, having a connecting port for delivery or removal of fluid, and a needle including: a proximal portion, including a first port of the lumen, and a distal portion, including a second port of the lumen; where the proximal portion of the needle is mounted to the support member so that the first port is fluidly connected to the connecting port; wherein the distal portion includes a sharpened tip provided on a first lengthwise side of the needle, and a deformable portion provided on an opposing, second lengthwise side of the needle, wherein the tip is configured to incise an entry point in the soft tissue of a patient as the tip is urged against the soft tissue by an urging force provided in a direction along the needle, and wherein the deformable portion is configured so that, in response to the urging force, the tip is deflectable away from the second lengthwise side of the needle as the tip is inserted into the soft tissue beyond the entry point.

According to another aspect of the disclosure, there is provided a needle insertion assembly including a support member and a base member; wherein the support member includes a connecting port, for delivery or removal of fluid, and a needle including: a proximal portion, including a first port of the lumen, and a distal portion, including a second port of the lumen; wherein the proximal portion of the needle is mounted to the support member so that the first port is fluidly connected to the connecting port; wherein the distal portion includes a sharpened tip provided on a first lengthwise side of the needle, and a deformable portion provided on an opposing, second lengthwise side of the needle, wherein the base member includes an engaging surface for contactingly engaging the soft tissue of a patient, the engaging surface of the base member having an opening, and a receiving portion configured to receivingly engage the support member so that the distal portion of the needle projects outward through the opening; wherein the tip of the distal portion of the needle is configured to incise an entry point in the soft tissue of a patient as the tip is urged against the soft tissue by an urging force provided in a direction along the needle, and wherein the deformable portion is configured so that, in response to the urging force, the tip is deflectable away from the second lengthwise side of the needle as the tip is inserted into the soft tissue beyond the entry point.

In certain embodiments, a complementary engaging element may be provide on the base member. Additionally, or alternatively, a support member may include a latch element configured to lockingly engage, preferably to selectively lockingly engage, with a complementary engaging element provide on the base member.

According to yet another aspect, a method of inserting a needle is provided, the needle having a lumen for distributing fluid into, or removing fluid from, a soft tissue of a patient, wherein the method includes providing a needle having a lumen, the needle includes a proximal portion and a distal portion, where the distal portion includes a sharpened tip provided on a first lengthwise side of the needle, and a deformable portion provided on an opposing, second lengthwise side of the needle; urging the tip is against the soft tissue with an urging force provided in a direction along the needle to incise an entry point in the soft tissue of a patient; and deflecting the tip away from the second lengthwise side of the needle as the tip is inserted into the soft tissue beyond the entry point.

In certain embodiments, the step of deflecting the tip away from the second lengthwise side of the needle may include deflecting the tip relative to the direction of the urging force at an angle in the range of from 20° to 40°, preferably 30°. Additionally, or alternatively, the step of deflecting the needle may provide an elastic or superelastic deformation of the deformable portion of the needle in a range of from greater than 0% to 9%, preferably a range of from greater than 0% to 6%.

According to a yet further aspect, a needle is provided, the needle enclosing a lumen for distributing fluid into, or removing fluid from, the body of a patient, the needle also includes: a proximal portion, including a first port of the lumen, and a distal portion, including a second port of the lumen; and a distal portion including a sharpened tip provided on a first lengthwise side of the needle, and a deformable portion provided on an opposing, second lengthwise side of needle; wherein the tip is configured to incise an entry point in the soft tissue of a patient as the tip is urged against the soft tissue by an urging force provided in a direction along the needle; and wherein the deformable portion is configured so that, in response to the urging force, the distal portion is moveable from a first condition, in which tip and the deformable portion are aligned in the direction of the urging force, to a second condition in which the tip is deflected laterally to the direction of the urging force.

In certain embodiments, in response to the urging force, the tip is deflected laterally to the direction of the urging force at an angle in the range of from 20° to 40°, preferably 30°.

Certain examples provide a needle inserter needle insertion assembly or a method that ensures the needle is inserted easily. In particular, the tip is urged to pierce the soft tissue with a consistent urging force. The tip is urged to pierce the soft tissue without requiring manual manipulation or orientation, improving reliability and repeatability of the insertion method.

Certain examples ensure the needle, particularly a flexible needle, is urged into the soft tissue with a controlled lateral deflection. In particular, an urging force provided directly along the longitudinal axis of the needle at the distal portion of the needle pierces the soft tissue with the sharpened tip at a predetermined angle.

Certain examples ensure a needle, particularly a flexible needle, ensures reliable flow of fluid through the needle lumen.

Certain examples ensure the tip of the needle, particularly the tip of a flexible needle, enters the soft tissue beyond the entry point at a predetermined angle.

Certain examples ensure the tip of the needle is secured within the needle inserter needle insertion assembly so that the needle inserter needle insertion assembly is convenient to use in a space-efficient manner.

### BRIEF DESCRIPTION OF THE DRAWINGS

Embodiments of the invention will now be described, by way of example only, with reference to the accompanying figures, in which:

FIGS. 1(a)-(c) are schematic partial views of a needle according to embodiments of the invention, consisting of (a) a perspective view, (b) a front view, and (c) a side view;

FIGS. 2(a)-(b) are schematic end views of respective needles according to embodiments of the invention, the needle of FIG. 2(b) being the needle of FIGS. 1(a)-(c);

FIG. 3 is a graph showing the temperature-induced phase transformation of nitinol between martensite and austenite phases;

FIG. 4 is a graph showing correlation of martensite start temperature Mₛ and the composition of nitinol;

FIG. 5A shows a side view, and FIG. 5B shows a cross-sectional view, of a needle according to another embodiment of the invention;

FIG. 6 shows a perspective view of the needle of FIG. 5A and FIG. 5B mounted to a support member;

FIG. 7A shows an upper perspective view, and FIG. 7B shows a lower perspective view, of a base member;

FIG. 8 shows a perspective view of a needle insertion assembly including the support member of FIG. 6 mounted to the base member of FIG. 7A and FIG. 7B:

FIG. 9 shows a cross-sectional view of the needle insertion assembly of FIG. 8:

FIG. 10 shows a side view of the needle insertion assembly of FIG. 8 prior to use:

FIG. 11 shows a side view of the needle insertion assembly of FIG. 8 in use:

FIG. 12 shows a graph illustrating certain properties of a nitinol material;

FIG. 13 shows a stress-strain diagram comparing certain properties of a needle formed from a nitinol material with those of a needle formed of stainless steel; and

FIG. 14 shows a graph illustrating certain properties of needles formed from a nitinol material.

### DETAILED DESCRIPTION OF EMBODIMENTS OF THE INVENTION

Certain embodiments of the invention may have particular application for use in medical devices for continuous subcutaneous insulin infusion (CSII) therapy, for example infusion sets. However, other applications are contemplated, such as cannula access ports and infusion pump systems, including patch pumps and so-called closed-loops systems, which are infusion pump systems used in combination with a continuous glucose monitoring (CGM) device to continually monitor blood sugar levels and adjust the amount of insulin delivered to a patient automatically. Certain embodiments of the invention have particular application for use in needles formed of a superelastic material, for example a nickel titanium alloy, i.e. nitinol.

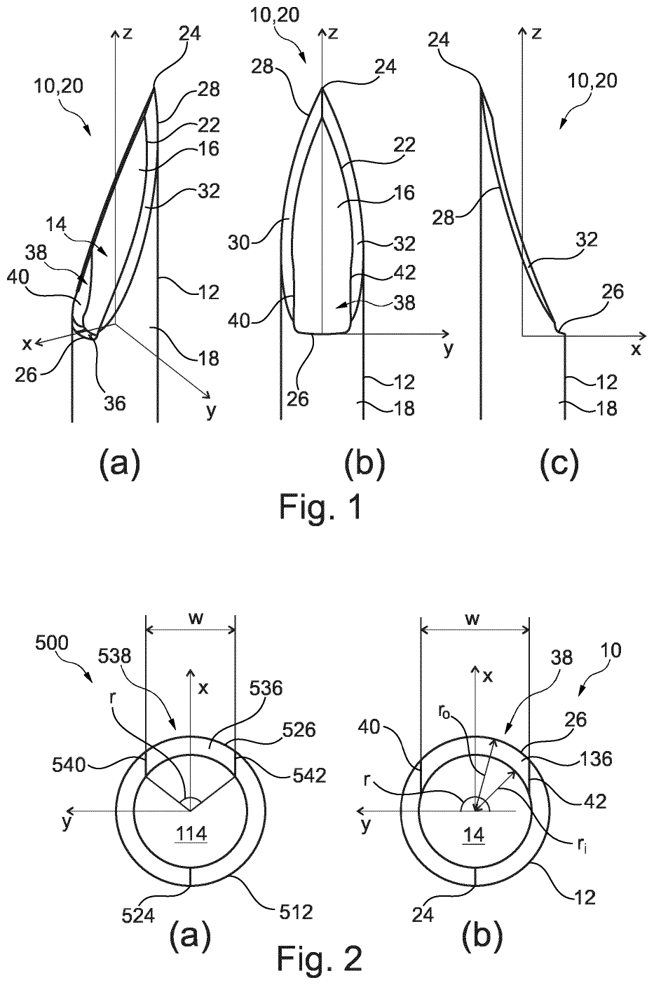

FIGS. 1(a)-(c) and 2(b) show a needle 10 according to an embodiment of the invention. The needle 10 is formed from a needle tube or cannula 12 defining a fluid carrying lumen 14. The needle tube 12 has inner surface 16 and an opposing outer surface 18 defining inner and outer radii rᵢ, rₒ of the needle tube 12, respectively. The difference between the inner and outer radii rᵢ, rₒ is the wall thickness of the needle tube 12. The needle 10 has a proximal end (not shown) for attachment to a medical device, for example an infusion set, such that the needle 10 is in fluid communication therewith. The needle 10 further has a distal end or tip 20 defining a fluid opening 22 for passage of a fluid to and/or from the lumen 14. The distal end 20 is formed to provide a point 24, which may be configured to facilitate insertion into patient tissue, and includes a heel 26 opposite the point 24. The distal end 20 also includes a cutting edge 28. As shown in the accompanying figures, an xyz coordinate system may be defined for the needle 10, the z-axis coinciding with a central axis of the lumen 14—the needle tube 12 therefore extending along the central axis between the proximal end and the distal end 20—and the x-axis passing through the centre of the heel 26.

The cutting edge 28 may be configured for cutting into patient tissue. In the illustrated embodiment, the cutting edge 28 is configured to provide the needle 10 with a lancet-type distal end 16 comprising two lancet surfaces 30, 32. As such, the surfaces 30, 32 coincide at the point 24 and are symmetrical either side thereof. In certain embodiments, including that illustrated in the accompanying figures, the cutting edge 28 may extend circumferentially, i.e. in a circumferential direction, about of the needle tube 12 and therefore about the opening 22 of the lumen 14. The cutting edge 28 may extend circumferentially continuously or discontinuously. The lancet surfaces 30, 32 may be planar or curved, and the exact configuration of the lancet surfaces 30, 32, including an angle of the surfaces 30, 32 with respect to the central axis of the needle tube 12, may be determined to provide a sharp cutting edge for any suitable configuration of the distal end 20.

The heel 26 extends radially between the inner and outer surfaces 16, 18 of the needle tube 12. In other words, the heel extends over the wall thickness of the needle tube 12. Crucially, at least a region of the heel 26 extends substantially orthogonal to the central axis—and therefore substantially orthogonal to the z-axis—to provide a non-cutting edge 34 of the distal end 20. Used herein, including in the appended claims, the term “non-cutting edge” is to be understood to mean an edge of the distal end 20 not intended for the purpose of cutting, for example not intended for the purpose of cutting patient tissue. By extending substantially orthogonal to the central axis, the heel 26 extends substantially at right angles to the inner and outer surfaces 16, 18 and provides a blunt region of the distal end or tip 20.

The substantially orthogonal region may be a substantially planar surface 36, i.e. the substantially orthogonal region may extend circumferentially about of the needle tube 12 as well as between the inner and outer surfaces 16, 18 of the needle tube 12. The extent to which the surface 36 extends about the needle tube 12 may be measured along the inner surface 16 thereof. As such, the surface 36 may extend over a radial angle γ measured along the inner surface 16 of the needle tube. The radial angle γ may be any suitable angle to prevent the heel 26 forming a cutting edge, and may vary depending upon the specific configuration of the distal end 20 of the needle 10. In certain embodiments, the radial angle γ may be at least approximately 30° or at least approximately 90°. In certain embodiments, the radial angle γ may be any angle up to approximately 180°. In certain embodiments, the radial angle γ may be at least approximately 90° and any angle up to approximately 180°. In certain embodiments, the radial angle γ may be approximately 180°, plus/minus 10° or 20°. Certain embodiments of the invention that have been found to work particularly well are those wherein the radial angle γ is approximately 180°, for example angles between 165° and 175°.

As shown in the accompanying figures, particularly FIG. 1(b), by the surface 36 extending about the needle tube 12, a cut-out or slot 38 may be formed in the needle tube 12. The cut-out 38 extends axially along the needle tube 12 from the opening 22 of the lumen 14. In such embodiments, the surface 36 forms a planar terminal end of the cut-out 38. However, certain embodiments are contemplated having a curved terminal end, wherein only a region, for example a central region of the heel 26, is orthogonal to the central axis of the needle tube 12.

The cut-out 38 is delimited in a direction parallel to the y-axis by a pair of opposing surfaces 40, 42 that each extend between the inner and outer radii rᵢ, rₒ of the needle tube 12. A distance between the opposing surfaces 40, 42 defines a width w of the cut-out 38. In certain embodiments, the width w may be equal to the inner diameter of the needle tube 12, i.e. twice the inner radius rᵢ. As in the illustrated embodiment, the opposing surfaces 40, 42 may be planar surfaces, and may additionally extend substantially parallel to one another, i.e. the opposing surfaces 40, 42 may extend in a direction substantially parallel to the x-axis. In embodiments wherein the opposing surfaces 40, 42 extend in a direction substantially parallel to the x-axis, the opposing surfaces 40, 42 may extend tangentially from the inner surface 16 of the needle tube 18. In such embodiments, the width w may be equal to the inner diameter of the needle tube 12 along the length of the cut-out 38. Certain embodiments of the invention that have been found to work particularly well are those wherein width w may be equal to the inner diameter of the needle tube 12 along the length of the cut-out 38.

FIG. 2(b) shows a needle 510 according to an alternative embodiment of the invention, with features the same or similar to those described above with reference to FIGS. 1(a)-(c) and 2(b) offset by a factor of 500. The difference between the embodiment of FIG. 2 and that of FIGS. 1(a)-(c) and 2(b) is that the width w is less than the diameter of the needle tube 512. As such, while the opposing surfaces 540, 542 extend in a direction substantially parallel to the x-axis, the surfaces 540, 542 do not extend tangentially from the inner surface 516 of the needle tube 516.

In certain embodiments, the inner surface 16 and/or outer surface 18 of the needle tube 12 may be configured to have desired properties, for example hydrophobic or hydrophilic properties. Particularly, it has been found beneficial that the outer surface 18 may be hydrophilic, at least more so relative to the inner surface 16, as this may facilitate adhesion to tissue and/or may reduce FBR, i.e. the human body is more prone to accept the presence of the needle 10 when the outer surface 18 is hydrophilic. It has also been found beneficial that the inner surface 16 may be hydrophobic, at least more so relative to the outer surface 18, as this may facilitate dispersion of the therapeutic agent by discouraging adhesion thereof to the inner surface 16 of the needle tube 12. Accordingly, in certain embodiments, the inner and/or outer surfaces 16, 18 may have a surface treatment to alter the hydrophobic/hydrophilic properties thereof, for example a surface treatment comprising one or more of a chemical treatment, an electrochemical treatment, polishing, etching or coating.

The size of the needle 10, including the length of the needle 10 and the inner and outer diameters of the needle tube 12, will vary depending upon the intended used of the particular embodiment. As the skilled reader will know, relatively longer needles, for example greater than 12 mm, may be used for intramuscular placement. Relatively shorter needles, for example less than 12 mm may be used for intravenous placement. Needles between 12 mm and 15 may be used for subcutaneous placement, including in certain embodiments of the needle 10 for CSII therapy. The needle 10 may have a wall thickness of at least 20 μm and/or no greater than 400 μm. Additionally, or alternatively, the needle 10 may have an outer diameter of at least 0.4 mm and/or no greater than 0.7 mm, and/or the needle 10 may have an inner diameter of at least 0.2 mm and/or no greater than 0.55 mm. In certain embodiments, the needle 10 may have an inner diameter of approximately 0.3 mm and/or an outer diameter of approximately 0.4 mm. In certain embodiments, the needle 10 may have an inner diameter of approximately 0.45 mm and/or an outer diameter of approximately 0.55 mm.

In certain embodiments, the cannula 12 may be superelastic, i.e. the cannula 12 may be formed of superelastic material, for example a superelastic alloy. Superelasticity, also referred to as pseudoelasticity, is an elastic response exhibited by certain materials to an applied stress. Superelasticity occurs when an applied stress induces an austenite to martensite phase transformation in the material and a corresponding strain, which is recoverable by removing the applied stress. Certain superelastic materials exhibit recoverable strains of up to 11%, which is significantly greater than more conventional materials. For example, 316 stainless steel (16% chromium, 10% nickel and 2% molybdenum), which is commonly used in medical applications, exhibits recoverable strains of approximately 0.5%. A superelastic alloy used in medical applications is nickel titanium alloy, commonly referred to as nitinol, as it exhibits exceptional biocompatibility. The cannula 12 may be formed of nitinol. As superelasticity is a stress-induced phase transformation from austenite to martensite, for the cannula 12 to exhibit optimum superelasticity it may be formed of so-called austenitic (or superelastic) nitinol, in which nitinol will be substantially fully austenitic, i.e. the primary crystalline structure of the alloy is austenite. Nitinol will remain substantially fully austenitic above its martensite start temperature Mₛ. This is important to note because an austenite to martensite phase transformation can be induced by cooling as well by applied stress.

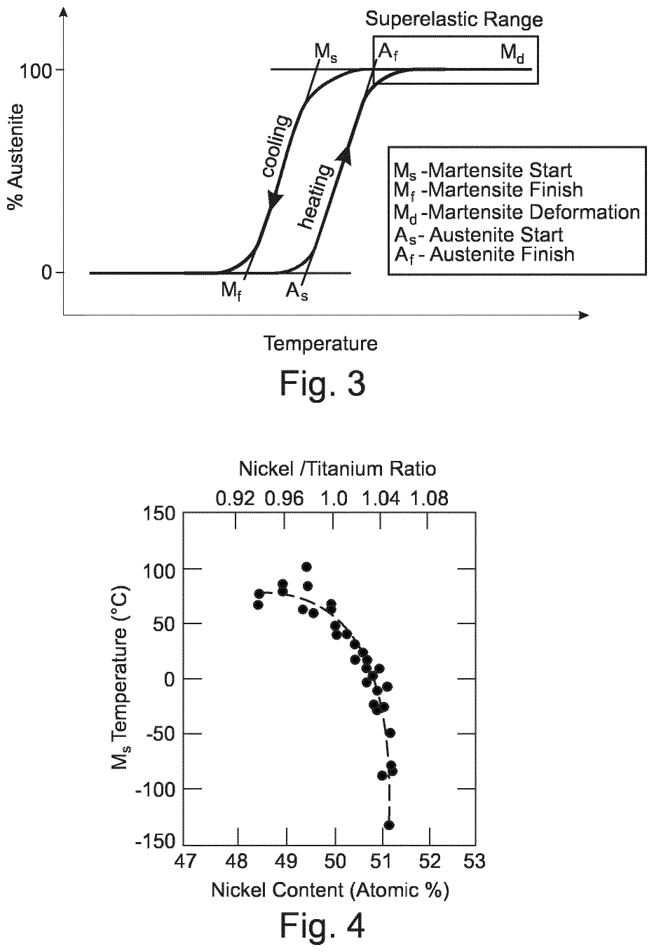

FIG. 3 shows the temperature-induced phase transformation of a nitinol alloy between the austenitic and martensitic phases, in which austenite is stable at relatively higher temperatures and martensite is stable at relatively lower temperatures. Heating nitinol beyond its austenite start temperature Aₛ causes it to transform to the austenitic phase. Nitinol will be substantially fully austenitic once heated above its austenite finish temperature A_{f}. As above, it is in this substantially fully austenitic phase that nitinol will exhibit optimum superelasticity, allowing the cannula 12 when formed of nitinol to elastically deform, i.e. flex/bend, through a relatively broad range of stresses without causing permanent deformation. The optimal superelastic range (also referred to as a superelastic window), between the austenite finish temperature A_{f} and the martensite deformation temperature M_{d}, is highlighted in FIG. 3. From the substantially fully austenitic phase, cooling nitinol beyond its martensite start temperature Mₛ causes nitinol to transition to the martensitic phase. Below its martensite finish temperature M_{f} nitinol will be substantially fully martensitic.

FIG. 3 also shows that nitinol exhibits thermal hysteresis, i.e. the temperature at which martensite transforms to austenite is not that at which austenite transforms to martensite. The hysteresis may be approximately 20 to 30° C. (i.e. A_{f}-M_{f}) for fully annealed nitinol alloys, such as those used in medical device applications. It is known that a greater thermal hysteresis will yield a greater mechanical hysteresis. The significance of its thermal hysteresis is that nitinol remains in its austenitic phase when cooled beyond its austenite finish temperature A_{f}. This means the cannula 12 when formed of austenitic nitinol will remain superelastic above its martensite start temperature Mₛ, which may therefore be the critical transformation temperature when selecting an alloy for forming the cannula 12.

FIG. 4 shows the correlation between the martensite start temperature Mₛ and the composition of nitinol. Since the martensite start temperature Mₛ is correlated with the nickel/titanium ratio it can be predetermined, for example a nitinol alloy can be selected having a martensite start temperature Mₛ from approximately −130° C. to approximately +110° C.

As above, certain embodiments of the invention may have particular application for use in devices for CSII therapy, in which the devices may be worn by a patient such that at least the tip 16 of the cannula 12 is placed in the patient's soft tissue. Due to the superelasticity of nitinol, the cannula 12 may reduce tissue damage by exhibiting a relatively high degree of flexibility. However, if the martensite percentage of the material forming the tip 16 begins to rise, exposure to stress may cause it undergo plastic deformation, resulting in an irregular, uncontrolled shape change. This may lead to tissue damage. While the patient's body heat may aid in maintaining nitinol in its austenitic phase, certain embodiments of the cannula 12 may be placed in the soft tissue of the patient to a relatively low depth, such as approximately 3 to 4 mm. As such, cold temperatures experienced by the patient may cause a temperature-induced phase transformation of the nitinol alloy forming the cannula 12 to its martensitic phase, i.e. if the martensite start temperature Mₛ is too high. Thus, in certain embodiments, the cannula 12 may be formed of a nitinol alloy selected to remain substantially fully austenitic (for example, at least 95% austenitic) in temperatures likely to be experienced by the patient to reduce the risk of the cannula 12 becoming martensitic.

As indicted in FIG. 3, superelasticity is exhibited up to the martensite deformation temperature M_{d}, which corresponds to the greatest temperature at which it is possible to stress-induce the formation of martensite. Above the martensite deformation temperature M_{d} the response to stress is non-elastic deformation of the austenitic microstructure, since martensite can no longer be formed, and thus permanent deformation. In other words, above the martensite deformation temperature M_{d} the nitinol will deform plastically and irreversibly.

The effective superelastic range of the nitinol may be increased to span more than 200° C., with a significant reduction in temperature-stress sensitivity, by subjecting the material to a controlled process. The nitinol may, for example, be subjected to an annealing process or treatment, e.g. an isobaric annealing process or treatment in Argon at approximately 350° C. to 400° C. In this way, the martensite deformation temperature M_{d} of the material is increased and the effective superelastic window or range may be increased or widened.

In view of the above, the cannula 12 may be formed of a nitinol alloy having been subjected to an isobaric annealing treatment in order to increase the effective superelastic window in order to ensure that elastic deformation through a relatively broad range of stresses is possible. In this way the cannula 12 can be manufactured from nitinol that elastically bends without permanent deformation of the cannula 12.

The cannula 12 may also be formed of a nitinol alloy having an austenite finish temperature A_{f} in the range of approximately −15° C. to approximately 20° C. in order to ensure that the cannula 12 has optimal or good superelastic properties at body temperature (approximately 37° C.).

More generally, the cannula 12 may be formed of a nitinol alloy selected such that the cannula 12, or part therefore, exhibits selected properties and/or characteristics with reference to a particular temperature to be experienced by the cannula 12. In certain embodiments, the particular temperature may be body temperature (approximately 37° C.) or room temperature (for example, approximately 20 to 22° C.).

FIG. 4 also shows that nitinol is typically composed of approximately 50 to 51% nickel by atomic percent, though as shown in FIG. 4 compositions outside this range are known, and various compositions may be suitable for forming the cannula 12. In certain embodiments, an atomic ratio of nickel to titanium is between 1.01 and 1.05. In certain embodiments, an atomic ratio of nickel to titanium is between 1.02 and 1.04.

The cannula 12 being formed of austenitic or superelastic nitinol may exhibit greater flexibility than a similarly-configured cannula formed of a more conventional material, such as stainless steel. The skilled reader will understand that the range of superelasticity for a particular composition of nitinol depends largely upon its nickel/titanium ratio. In certain embodiments, the cannula 12 may be formed of nitinol having a nickel/titanium ratio selected to provide the cannula 12 with particular properties and/or characteristics. The skilled reader will also understand that the range of superelasticity of the nitinol also depends on how the material has been processed. In certain embodiments, the cannula 12 may be formed of nitinol that has been processed or treated in a controlled way in order to provide the cannula 12 with particular properties and/or characteristics.

Manufacture of the needle 10 will depend upon the material selected from which to form the needle 10. In certain embodiments, including embodiments wherein the needle 10 is formed of nitinol, the distal end 20—including for example the point 24, the heel 26 and the cutting edge 28—may be formed using a thermal based laser process. Such processes are known to be used for cutting nitinol and utilise high intensity focused light to locally melt the material to form a cut, wherein the molten material is subsequently driven out from/through the cut using a supply of pressurised gas. In certain embodiments, femtosecond laser processes can be used for cutting nitinol, and these processes involve high power, short laser pulses that cause direct solid to plasma ablation of nitinol.

The invention is not restricted to the details of any foregoing embodiments. For example, while the needle 10 as shown in the illustrated embodiment is configured to as a lancet needle, other needle types are contemplated, for example a bias bevel needle or a back-cut bevel needle.

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

As used herein, elastic and superelastic deformation refers to the recoverable deformation of at least a portion of a needle so that the portion returns from a deformed, energised state to an un-deformed state. As used herein, the needle axis may be the central longitudinal axis of the needle.

In certain embodiments, an elastic deformation gives a lengthwise extension, or a lengthwise compression, of a portion of the needle along a linear axis, particularly along the needle axis. That is, in the un-deformed state, the portion of the needle is disposed along a needle axis and, in the energised state, the portion of needle is elastically extended, or elastically compressed, along the needle axis.

In certain embodiments, an elastic deformation gives a deformation of a portion of the needle to curve the needle. That is, in the un-deformed state, the portion of the needle lies along a needle axis and, in the energised state, the portion of the needle is deformed away from the needle axis. In this way, a region of the needle on an outside of the curve that is caused by deforming the needle is extended elastically. Another region of the needle on an inside of the curve that is caused by deforming the needle is compressed elastically.

As used herein, a deformation limit refers to a limit of deformation of at least a portion of the needle when it is elastically deformed lengthwise along a linear axis and/or a limit of deformation of a portion of a needle that is elastically deformed to form a curve. The deformation limit is thereby the limit of any localised elastic extension or localised elastic compression within the region of the curved portion of the needle.

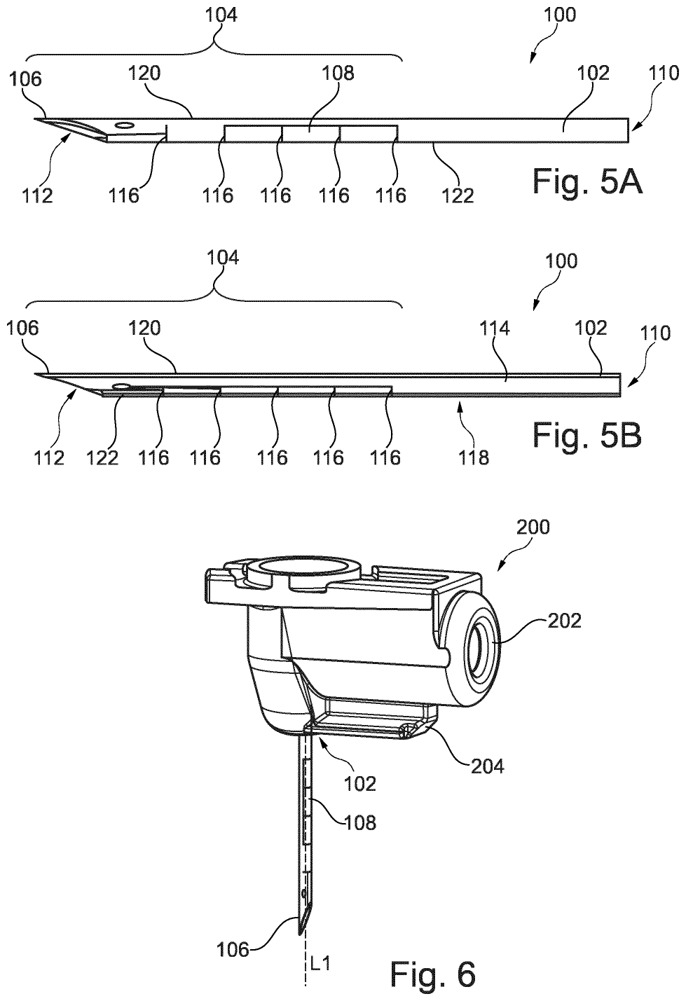

Referring now to FIG. 5A and FIG. 5B, there is shown a needle 100, having a lumen 114 for distributing fluid into, or removing fluid from, a soft tissue of a patient, the needle 100 includes: a proximal portion 102, including a first port 110 of the lumen 114, and a distal portion 104, including a second port 112 of the lumen 114. The distal portion 104 includes a sharpened tip 106 provided on a first lengthwise side 120 of the needle 100 and a deformable portion 108 provided on an opposing, second lengthwise side 122 of the needle 100. The tip 106 is configured to incise an entry point in the soft tissue of a patient as the tip 106 is urged against the soft tissue by an urging force provided in a direction along the needle 100. The deformable portion 108 is configured so that, in response to the urging force, the tip 106 is deflectable away from the second lengthwise side 122 of the needle as the tip is inserted into the soft tissue beyond the entry point.

The lumen 114 of the needle 100, particularly the lumen 114 within the distal portion 104, includes a circular cross-section extending within the needle 100. That is, along at least the distal portion 104, in an un-deformed state, the lumen 114 forms a cylindrical tube. The first lengthwise side 120 of the needle and the second lengthwise side 122 of the needle are provided on opposing sides of the cross-section. In the embodiment shown, the first lengthwise side 120 extends along the needle 100 from the tip 106 to the proximal portion 102. The second lengthwise side 122 extends along the needle 100 to the proximal portion 102 from a region diametrically opposed to the tip 106 of the distal portion 104. Stated differently, with the needle 100 in a straight condition, the first and second lengthwise sides extend along the needle 100 on opposing axial lengths of an outer surface 118 of the needle 100.

The deformable portion 108 of the needle 100 includes a plurality of apertures 116 extending through the needle 100. Each aperture 116 extends from the outer surface 118 of the distal portion 104 to the lumen 114. The lumen 114 is circumscribed by the needle wall. Each aperture 116 extends through the needle wall.

Each aperture 116 is an elongate slot oriented to extend partially around a circumference of the distal portion 104. In the embodiment shown, each elongate slot is substantially of equal length and oriented to extend circumferentially, that is perpendicular to the longitudinal axis of the needle 100 in the region of the respective aperture 116. In the embodiment shown, each elongate slot extends substantially 180° around the circumference of the distal portion.

As will be apparent, other suitable shape, length or orientation of apertures may be provided in the deformable portion 108. In certain embodiments, the plurality of apertures may each be of varying length. Certain apertures of the plurality of apertures may extend further around the circumference than other apertures. In certain embodiments, each aperture may be any suitable shape, such as a lozenge shape, ellipse shape. A primary lengthwise axis of each aperture may be oriented at an angle to the longitudinal axis of the needle in the deformable portion 108, for example, oriented at an angle in a range of from 30° to 180°, preferably in a range of from 45° to 150°, more preferably in a range of from 60° to 120°. Certain configurations may include a plurality of helical elongate slots. Certain apertures may be oriented at an acute angle to the longitudinal axis of the needle 100 in the region of the respective aperture 116. Each aperture 116 of the plurality of apertures is spaced apart along the deformable portion 108 in the lengthwise direction of the needle 100. The apertures 116 are laterally arranged with a spacing of 0.8 mm.

The deformable portion 108 extends along the distal portion 104 for a distance of 4 mm. That is, the deformable portion 108 spans 4 mm along the longitudinal axis of the needle 100 with the plurality of apertures arranged within the span of 4 mm. The apertures are spaced evenly so that each aperture 116 is spaced 0.8 mm apart from neighbouring apertures 116.

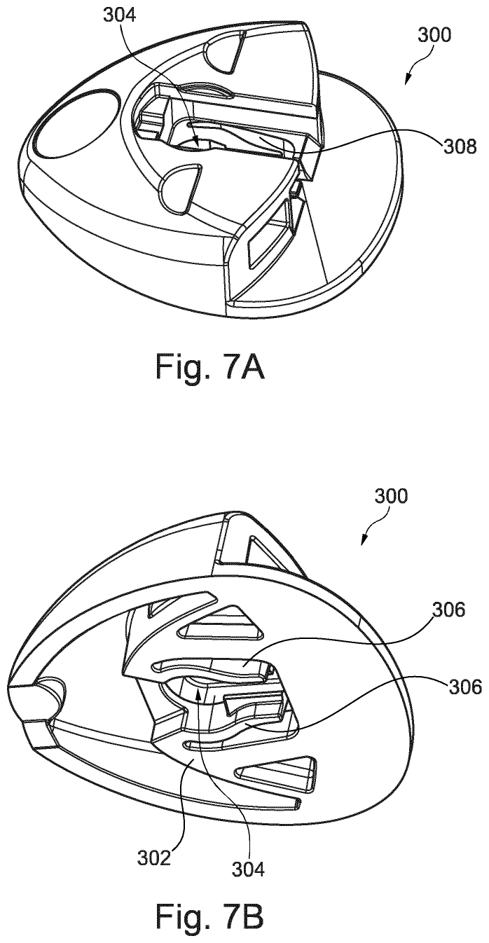

Referring now to FIG. 6, there is shown a support member 200 of a needle insertion assembly including the needle 100 as described with reference to FIG. 5A and FIG. 5B. The support member 200 includes a connecting port 202 for delivery or removal of fluid to the needle 100. The needle 100 is arranged so that its proximal portion is mounted to the support member 200 with the first port is fluidly connected to the connecting port. The needle 100 is mounted to the support member 200 ready for use, so that the needle 100 extends away from a lower surface of the support member 200 along a longitudinal axis L1. The support member 200 includes a latch element 204. The latch element 204 is configured to lockingly engage with a complementary engaging element 306 provide on a base member 300, as described with reference to FIG. 7B.

Referring now to FIG. 7A and FIG. 7B, there is shown a base member 300 of a needle insertion assembly. The base member 300 includes a receiving portion 308, configured to receivingly engage the support member 200 described with reference to FIG. 6, as well as an engaging surface 302 for contactingly engaging the soft tissue of a patient. The engaging surface 302 includes an opening 304. The opening extends through the base member 300 from the engaging surface 302 to the receiving portion 308. The receiving portion 308 includes a wall portion defining a cavity leading from an upper surface of the base member 300 to the opening 304. The receiving portion 308 is of suitable shape and dimensions to receive the support member 200. An engaging element 306 is provided in a lower portion of the receiving portion 308, proximal to the opening 304.

To use the needle 100, the support member 200 is assembled with the receiving portion 308 of the base member 300. The support member 200 is loaded into the base member 300 so that the distal portion 104 is inserted, first, through the receiving portion 308, and then through the opening 304. In this way, the receiving portion 308 receivingly engages the support member 200 so that the distal portion 104 of the needle 100 projects outward through the opening 304.

The engaging element 306 includes a pair of deformable tangs. The latch element 204 of the support member 200 includes a profiled ridge with a cam surface. The cam surface is arranged to deflect the tangs of the engaging element 306 away from one another as the support member 200 engages the base member 300, so that the latch element 204 is lock to the engaging element 306. The support member 200 is mounted to the base member 300 ready for use so that the longitudinal axis L1 of the needle 100 extends away from the engaging surface 302 of the base member 300. In particular, in the embodiment shown, the longitudinal axis L1 is oriented to be perpendicular to the engaging surface 302 when the support member 200 is lockingly engaged with the base member 300 of the needle insertion assembly 400.

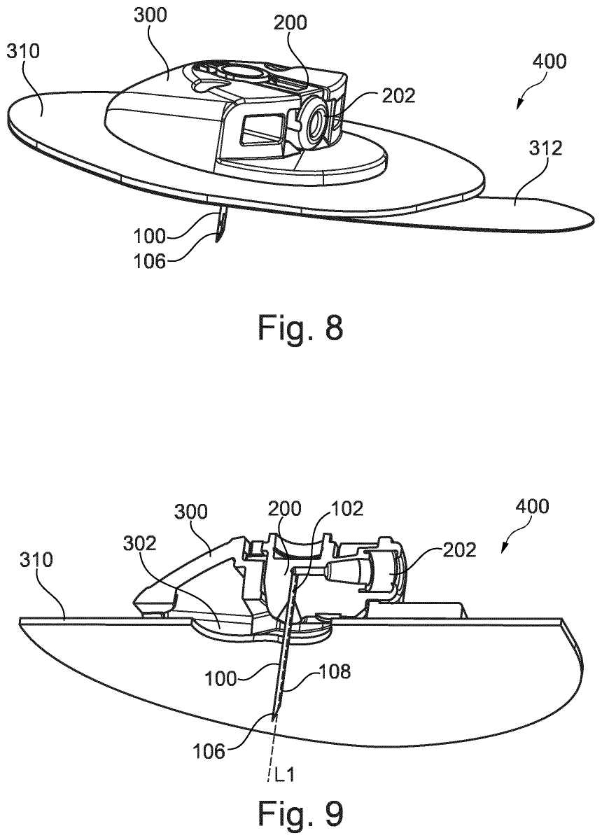

Referring now to FIG. 8 and FIG. 9, there is shown a needle insertion assembly 400 including the support member 200 of FIG. 6 assembled with the base member 300 if FIG. 7A and FIG. 7B. The needle insertion assembly 400 is shown in an assembled configuration, with the support member 200 lockingly engaged with the base member 300, but without affixing the needle insertion assembly 400 to soft tissue.

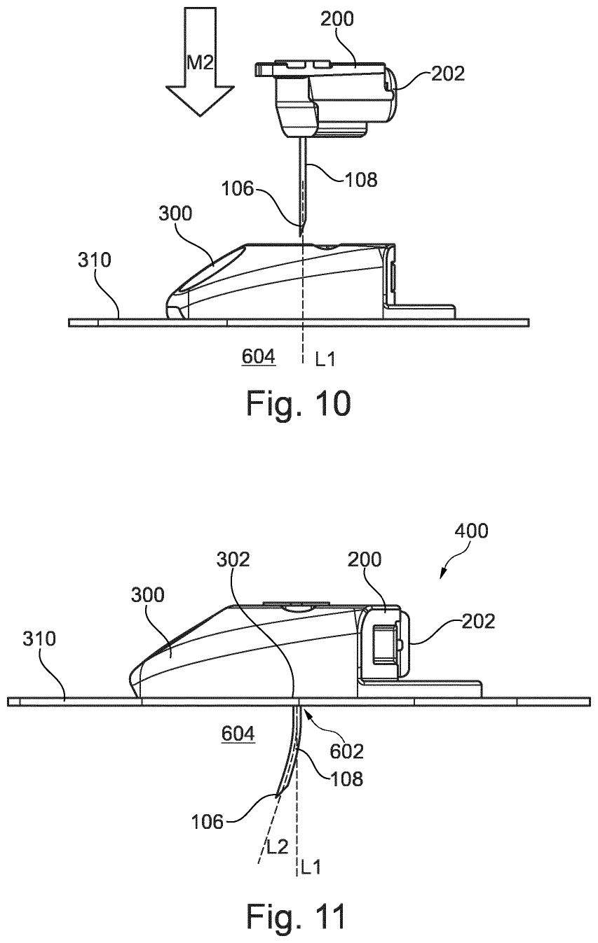

The respective method of using the needle insertion assembly 400 is described herein with reference to FIG. 10 and FIG. 11. The needle insertion assembly 400 includes an adhesive patch 310. The adhesive patch 310 is suitably tacky to affix a lower surface of the base member 300, typically the engaging surface 302, to the soft tissue of the patient. With the needle insertion assembly 400 affixed to the patient, the support member 200, including the needle 100, may be mounted to the needle insertion assembly 400 as described herein, so that the needle 100 is inserted into the soft tissue of the patient. As will appreciated, the needle insertion assembly 400 may be affixed or otherwise secured to the patient without an adhesive patch 310, instead using other suitable means.

In the embodiment shown, as shown particularly in FIG. 8, a removable strip 312 is located over the adhesive patch 310. The removable strip 312 is removed from the adhesive patch 310 when the base member 300 is to be affixed to the soft tissue. The adhesive patch 310 includes through-hole. The adhesive patch 310 is suitably positioned to align the through-hole with the opening 304 on the engaging surface 302 of the base member 300 when the adhesive patch 310 is affixed to the lower surface. The needle insertion assembly 400 may optionally include a protective cover (not shown) mountable to, or around, a perimeter surface of the base member 300. The protective cover may be mounted to the base member 300 after the base member 300 is affixed to a patient's soft tissue, enclosing perimeter surface of the base member 300 against the soft tissue. The protective cover thereby reduces ingress of dirt or contamination between the soft tissue and the needle insertion assembly 400 during use.

Referring now to FIG. 10 and FIG. 11, the needle insertion assembly 400 is shown in use. Referring specifically, to FIG. 10, the needle insertion assembly 400 is shown affixed to the soft tissue 604 of a patient using the adhesive patch 310. The support member 200 is disengaged from the base member 300. The needle 100 is in a first condition, in which tip and the deformable portion are aligned along the longitudinal axis L1. The support member 200 is positioned for engagement with the base member 300. That is, the support member 200 is disposed above base member 300 so that the longitudinal axis L1 is aligned perpendicular to the engaging surface 302 of the base member 300. In practice, this means that the longitudinal axis L1 is perpendicular to the soft tissue 604 to which the needle insertion assembly 400 is affixed. The support member 200 is moved into engagement with the base member 300 by inserting support member 200 into the receiving portion 308 of the base member 300. The distal portion 104 of the needle 100 is inserted through the opening 304 so that the tip 106 on the first lengthwise side 120 of the needle 100 contacts the surface of the soft tissue 604. The needle 100 is thereby positioned to incise an entry point 602 in the soft tissue 604. The support member 200 is engaged with the base member 300 by moving the support member 200 into receiving engagement with the receiving portion 308. In particular, the support member 200 is urged into the receiving portion 308 in the direction of the longitudinal axis L1. The latch element 204 is urged into locking engagement with the engaging element 306 of the base member 300.

Moving the support member 200 into receiving engagement with the receiving portion 308 applies an urging force along the longitudinal axis L1 of the needle 100. The deformable portion 108 on the second lengthwise side 122 of the needle 100 responds to the urging force to deform so that tip 106 of the needle 100 is deflected away from the second lengthwise side 122 as the tip passes beyond the entry point 602 on the soft tissue 604. The needle 100 is deflected towards an insertion axis L2. The insertion axis L2 is disposed at an acute angle to the longitudinal axis L1 of the needle. The tip 106 is thereby inserted into the soft tissue 604 along the insertion axis L2. The deformable portion 108 is configured so that each aperture 116 is openable as the tip 106 is deflected. That is, the apertures 116 open in response to the urging force. Stated differently, the deformable portion is configured to move in response to the urging force from a compressed condition, in which apertures have a minimal open area, to an expanded condition, in which the apertures have a maximal open area. Due to the location of apertures 116 on the second lengthwise side 122, the expansion to an expanded condition deflects the tip 106 of the distal portion 104 away from the second lengthwise side 122, that is laterally away from the longitudinal axis L1.

In the embodiment shown, the tip 106 is deflected in response to an urging force of 2.6N. The deformable portion is configured so that, in response to the urging force, the tip is deflected relative to the direction of the urging force at an angle of 30°. In other words, as the tip passes into the soft tissue 604 beyond the entry point 602, the tip deflects towards the insertion axis L2, to be disposed at angle, typically an angle of 30°, relative to the longitudinal axis L1. The needle 100 and needle insertion assembly 400 described herein provide a convenient method of inserting a needle having a lumen for distributing fluid into, or removing fluid from, a soft tissue of a patient.

According to a first step, the method includes providing a needle having a lumen, the needle including a proximal portion and a distal portion, wherein the distal portion includes: a sharpened tip provided on a first lengthwise side 120 of the needle, and a deformable portion provided on an opposing, second lengthwise side 122 of the needle. According to another step, the method includes urging the tip is against the soft tissue with an urging force provided in a direction along the needle to incise an entry point in the soft tissue of a patient. According to a further step, the method includes deflecting the tip away from the second lengthwise side 122 of the needle as the tip is inserted into the soft tissue beyond the entry point. In the embodiment shown, the method includes deflecting the tip away from the second lengthwise side 122 of the needle at an angle of 30°.

In certain embodiments, the needle may be superelastic, i.e. the needle may be formed from a superelastic material, for example a superelastic alloy. Superelasticity, also referred to as pseudo-elasticity, is an elastic response exhibited by certain materials to an applied stress. Superelasticity occurs when an applied stress induces an austenite to martensite phase transformation in the material and a corresponding strain, which is recoverable by removing the applied stress. Certain superelastic materials exhibit recoverable strains of up to 11%, which is significantly greater than more conventional materials. For example, 316 stainless steel (16% chromium, 10% nickel and 2% molybdenum), which is commonly used in medical applications, exhibits recoverable strains of approximately 0.5%. A superelastic alloy used in medical applications is nickel titanium alloy, commonly referred to as nitinol, as it exhibits exceptional biocompatibility. The needle may be formed of nitinol. As superelasticity is a stress-induced phase transformation from austenite to martensite, for the needle to exhibit optimum superelasticity it may be formed of so-called austenitic (or superelastic) nitinol, in which nitinol will be substantially fully austenitic, i.e. the primary crystalline structure of the alloy is austenite. Nitinol will remain substantially fully austenitic above its martensite start temperature Mₛ. This is important to note because an austenite to martensite phase transformation can be induced by cooling as well by applied stress.

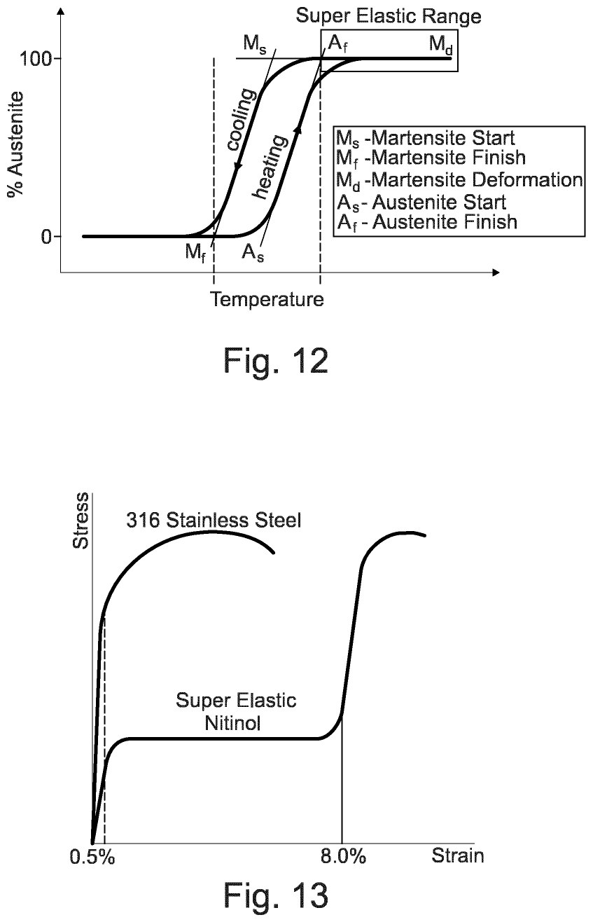

Referring now to FIG. 12, there is shown a temperature-induced phase transformation of a nitinol alloy between the austenitic and martensitic phases, in which austenite is stable at relatively higher temperatures and martensite is stable at relatively lower temperatures. Heating nitinol beyond its austenite start temperature Aₛ causes it to transform to the austenitic phase. Nitinol will be substantially fully austenitic once heated above its austenite finish temperature A_{f}. As above, it is in this substantially fully austenitic phase that nitinol will exhibit optimum superelasticity, allowing the needle when formed of nitinol to elastically deform, i.e. flex/bend, through a relatively broad range of stresses without causing permanent deformation. The optimal superelastic range (also referred to as a superelastic window), between the austenite finish temperature A_{f} and the martensite deformation temperature M_{d}, is highlighted in FIG. 12. From the substantially fully austenitic phase, cooling nitinol beyond its martensite start temperature Mₛ causes nitinol to transition to the martensitic phase. Below its martensite finish temperature M_{f} nitinol will be substantially fully martensitic.

The graph in FIG. 12 also shows that nitinol exhibits thermal hysteresis, i.e. the temperature at which martensite transforms to austenite is not that at which austenite transforms to martensite. The hysteresis may be approximately 20 to 30° C. (i.e. A_{f}-M_{f}) for fully annealed nitinol alloys, such as those used in medical device applications. It is known that a greater thermal hysteresis will yield a greater mechanical hysteresis. The significance of its thermal hysteresis is that nitinol remains in its austenitic phase when cooled beyond its austenite finish temperature A_{f}. This means the needle when formed of austenitic nitinol will remain superelastic above its martensite start temperature Mₛ, which may therefore be the critical transformation temperature when selecting an alloy for forming the needle.

Referring now to FIG. 13, there is shown a graph comparing the behavior of a needle formed of 316 stainless steel with a needle formed of a nitinol material. As is evident from the figure, the nitinol needle has a large range of elastic deformation in which increased strain does not significantly increase the stresses within the needle. In the illustrated form, stresses begin to rise at about 8% strain. As such, it may be advisable to maintain the strain associated with the needle below about 6% in order to ensure that the needle remains within its elastic deformation range. That is, it may be advisable to maintain the strain associated with the needle below about 6% in order to ensure that the needle remains below its elastic deformation limit.

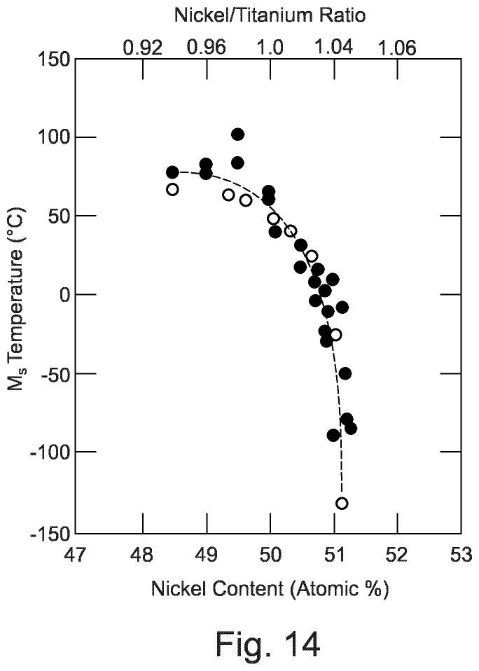

Referring now to FIG. 14, there is shown a correlation between the martensite start temperature Mₛ and the composition of nitinol. Since the martensite start temperature Mₛ is correlated with the nickel/titanium ratio it can be predetermined, for example a nitinol alloy can be selected having a martensite start temperature Mₛ from approximately −130° C. to approximately +110° C.

As above, certain embodiments of the invention may have particular application for use in devices for CSII therapy, in which the devices may be worn by a patient such that at least the tip and the distal portion of the needle is placed in the patient's soft tissue. Due to the superelasticity of nitinol, the needle may reduce tissue damage by exhibiting a relatively high degree of flexibility. However, if the martensite percentage of the material forming the tip 16 begins to rise, exposure to stress may cause it undergo plastic deformation, resulting in an irregular, uncontrolled shape change. This may lead to tissue damage. While the patient's body heat may aid in maintaining nitinol in its austenitic phase, certain embodiments of the needle may be placed in the soft tissue of the patient to a relatively low depth, such as approximately 3 to 4 mm. As such, cold temperatures experienced by the patient may cause a temperature-induced phase transformation of the nitinol alloy forming the needle to its martensitic phase, i.e. if the martensite start temperature Mₛ is too high. Thus, in certain embodiments, the needle may be formed of a nitinol alloy selected to remain substantially fully austenitic (for example, at least 95% austenitic) in temperatures likely to be experienced by the patient to reduce the risk of the needle becoming martensitic.

As indicated in FIG. 14, superelasticity is exhibited up to the martensite deformation temperature M_{d}, which corresponds to the greatest temperature at which it is possible to stress-induce the formation of martensite. Above the martensite deformation temperature M_{d} the response to stress is non-elastic deformation of the austenitic microstructure, since martensite can no longer be formed, and thus permanent deformation. In other words, above the martensite deformation temperature M_{d} the nitinol will deform plastically and irreversibly.

The effective superelastic window of the nitinol may be increased to span more than 200° C., with a significant reduction in temperature-stress sensitivity, by subjecting the material to a controlled process. The nitinol may, for example, be subjected to an annealing process or treatment, e.g. an isobaric annealing process or treatment in Argon at approximately 350° C. to 400° C. In this way, the martensite deformation temperature M_{d} of the material is increased and the effective superelastic window or range may be increased or widened.

In view of the above, the needle may be formed of a nitinol alloy having been subjected to an isobaric annealing treatment in order to increase the effective superelastic window in order to ensure that elastic deformation through a relatively broad range of stresses is possible. In this way the needle can be manufactured from nitinol that elastically bends without permanent deformation of the needle. The needle may also be formed of a nitinol alloy having an austenite finish temperature A_{f} in the range of approximately −15° C. to approximately 20° C. in order to ensure that the needle has optimal or good superelastic properties at body temperature (approximately 37° C.).

More generally, the needle may be formed of a nitinol alloy selected such that the needle, or part therefore, exhibits selected properties and/or characteristics with reference to a particular temperature to be experienced by the needle. In certain embodiments, the particular temperature may be body temperature (approximately 37° C.) or room temperature (for example, approximately 20 to 22° C.).

FIG. 8 also shows that nitinol is typically composed of approximately 50 to 51% nickel by atomic percent, though as shown in FIG. 8 compositions outside this range are known, and various compositions may be suitable for forming the needle. In certain embodiments, an atomic ratio of nickel to titanium is between 1.01 and 1.05. In certain embodiments, an atomic ratio of nickel to titanium is between 1.02 and 1.04.

The needle being formed of austenitic or superelastic nitinol may exhibit greater flexibility than a similarly-configured cannula formed of a more conventional material, such as stainless steel. The skilled reader will understand that the range of superelasticity for a particular composition of nitinol depends largely upon its nickel/titanium ratio. In certain embodiments, the needle may be formed of nitinol having a nickel/titanium ratio selected to provide the needle with particular properties and/or characteristics. The skilled reader will also understand that the range of superelasticity of the nitinol also depends on how the material has been processed. In certain embodiments, the needle may be formed of nitinol that has been processed or treated in a controlled way in order to provide the needle with particular properties and/or characteristics.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. In particular, the phrase “certain embodiments” is to be understood to mean any embodiment described, illustrated, or otherwise disclosed herein. All of the features disclosed in this specification (including any accompanying claims, abstract and drawings), and/or all of the steps of any method or process so disclosed, may be combined in any combination, except combinations where at least some of such features and/or steps are mutually exclusive. The invention extends to any novel one, or any novel combination, of the features disclosed in this specification (including any accompanying claims, abstract and drawings), or to any novel one, or any novel combination, of the steps of any method or process so disclosed.

Attention is directed to all papers and documents which are filed concurrently with or previous to this specification in connection with this application and which are open to public inspection with this specification, and the contents of all such papers and documents are incorporated herein by reference.

1. A needle comprising a needle tube extending along a central axis between proximal and distal ends of the needle, the needle tube defining a lumen, and the distal end defining an opening into the lumen and comprising:
a point,
a cutting edge, and
a heel extending between inner and outer surfaces of the needle tube, at least a region of the heel extending substantially orthogonally to the central axis to provide a non-cutting edge of the distal end.

2. A needle according to claim 1, wherein the substantially orthogonal region is a first substantially planar surface extending over a radial angle measured along an inner surface of the needle tube.

3. A needle according to claim 2, wherein the radial angle is at least approximately 30° and/or up to approximately 180°.

4. A needle according to any preceding claim, wherein the cutting edge comprises a second surface extending from at least one side of the needle tip.

5. A needle according to claim 4, wherein the cutting edge comprises a third surface, and each of the second and third surfaces extend from a respective side of the needle tip.

6. A needle according to claim 5, wherein the second and third surfaces extend symmetrically to one another on their respective side of the needle tip.

7. A needle according to any of claims 4 to 6, wherein the second and/or third surfaces are substantially planar surfaces.

8. A needle according to any preceding claim, wherein the point is diametrically opposite the centre of the heel.

9. A needle according to any proceeding claim, wherein the heel extends about the needle tube to form a cut-out therein extending substantially axially from the opening.

10. A needle according to claim 9, wherein the cut-out has a width equal to the inner diameter of the needle tube.

11. A needle according to claim 9 or 10, wherein the cut-out is delimited by opposing fourth and fifth substantially planar surfaces extending between the inner and outer surfaces of the needle tube.

12. A needle according to claim 11, wherein at least one of the fourth and fifth substantially planar surfaces extends tangentially from the inner surface of the needle tube.

13. A needle according to any preceding claim, wherein the needle is formed of a superelastic material, for example a nickel titanium alloy.

14. A method of manufacturing a needle, the method comprising:
cutting a needle tube to form an end thereof comprising a point, a cutting edge and a heel,
wherein cutting the needle tube to form the heel includes cutting a region of the heel to extend between inner and outer surfaces of the needle tube substantially orthogonal to a central axis of the needle tube.

15. A method according to claim 14, wherein cutting is laser cutting, for example thermal laser cutting and/or femtosecond laser cutting.

16. A needle having a lumen for distributing fluid into, or removing fluid from, a soft tissue of a patient, the needle comprising:
a proximal portion, including a first port of the lumen, and a distal portion, including a second port of the lumen;
wherein the distal portion includes:
a sharpened tip provided on a first lengthwise side of the needle; and
a deformable portion provided on an opposing, second lengthwise side of the needle;
wherein the tip is configured to incise an entry point in the soft tissue of a patient as the tip is urged against the soft tissue by an urging force provided in a direction along the needle; and
wherein the deformable portion is configured so that, in response to the urging force, the tip is deflectable away from the second lengthwise side of the needle as the tip is inserted into the soft tissue beyond the entry point.

17. The needle according to claim 16, wherein the tip is deflectable in response to an urging force in a range of from 1 N to 5 N, preferably from 2 N to 4 N, more preferably from 2.5 N to 3 N.

18. The needle according to claim 16 or 17, wherein the deformable portion includes at least one aperture extending through the needle from an outer surface of the distal portion to the lumen.

19. The needle according to any one of claims 16 to 18, wherein the at least one aperture is an elongate slot oriented to extend partially around a circumference of the distal portion.

20. The needle according to claim 19, wherein each elongate slot extends around the circumference of the distal portion in a range of from 30° to 180°, and optionally in a range of from 45° to 150° or in a range of from 60° to 120°.

21. The needle according to any one of claims 16 to 20, wherein the at least one aperture is a plurality of apertures spaced apart along the lumen in the lengthwise direction of the needle with a spacing in a range of from 0.5 mm to 2 mm.

22. The needle according to any one of claims 16 to 20, wherein the deformable portion extends along the distal portion for a distance in a range of from 2 mm to 20 mm, and optionally in a range of from 3 mm to 10 mm.

23. The needle according to any one of claims 16 to 22, wherein the deformable portion is configured so that in response to the urging force the distal portion is deformed up to 6%.

24. The needle according to any one of claims 16 to 23, wherein the deformable portion is configured so that, in response to the urging force, the tip is deflected relative to the direction of the urging force at an angle in the range of from 20° to 40°, and optionally 30°.

25. The needle according to any one of claims 16 to 24, wherein at least in the distal portion, the lumen comprises a circular cross-section extending within the needle, and wherein the first lengthwise side of the needle and the second lengthwise side of the needle are provided on opposing sides of the cross-section.

26. The needle according to any one of claims 16 to 25, wherein the needle is elastically deformable or superelastically deformable.

27. The needle according to claim 26, wherein the needle is elastically deformable or superelastically deformable in a range of from greater than 0% to 9%, preferably a range of from greater than 0% to 6%.

28. The needle according to any one of claims 16 to 27, wherein the needle is formed from a pseudo-elastic material or a superelastic material.

29. The needle according to claim 28, wherein the needle is formed from a nitinol material, typically a nitinol material with an austenite finish temperature in the range of from −15° C. to 30° C., preferably in the range of from 0° C. to 20° C.

30. A needle according to any one of claims, 16 to 29 wherein the needle has one or more of:
a wall thickness defined between an outer surface and a lumen surface, wherein the wall thickness is at least 20 μm;
a wall thickness defined between an outer surface and a lumen surface, wherein the wall thickness is less than 400 μm;
an outer diameter, wherein the outer diameter is less than 0.7 mm;
an outer diameter, wherein the outer diameter is at least 0.4 mm;
an inner diameter, wherein the inner diameter is at least 0.2 mm; and
an inner diameter, wherein the inner diameter is less than 0.5 mm.

31. The needle according to any one of claims 16 to 30, wherein the deformable portion is deflectable around an arcuate path with a pitch radius of at least 3.5 mm.

32. A needle insertion assembly comprising:
a support member, comprising a connecting port for delivery or removal of fluid; and
a needle according to any one of claims 1 to 16;
wherein the proximal portion of the needle is mounted to the support member so that the first port is fluidly connected to the connecting port.

33. A needle insertion assembly according to claim 32, further comprising a base member comprising:
an engaging surface for contactingly engaging the soft tissue of a patient, wherein the engaging surface includes an opening; and
a receiving portion configured to receivingly engage the support member so that the distal portion of the needle projects outward through the opening.

34. A needle insertion assembly according to claim 33, wherein the support member comprises a latch element configured to lockingly engage, preferably to selectively lockingly engage, with a complementary engaging element provide on the base member.

35. A method of inserting a needle having a lumen for distributing fluid into, or removing fluid from, a soft tissue of a patient, the method comprising:
providing a needle having a lumen, the needle comprising a proximal portion and a distal portion, wherein the distal portion comprises:
a sharpened tip provided on a first lengthwise side of the needle; and
a deformable portion provided on an opposing, second lengthwise side of the needle;
urging the tip is against the soft tissue with an urging force provided in a direction along the needle to incise an entry point in the soft tissue of a patient; and
deflecting the tip away from the second lengthwise side of the needle as the tip is inserted into the soft tissue beyond the entry point.

36. The method according to claim 35, wherein the step of deflecting the tip away from the second lengthwise side of the needle comprises deflecting the tip relative to the direction of the urging force at an angle in the range of from 20° to 40°, preferably 30°.

37. The method according to claim 35, wherein the step of deflecting the needle provides an elastic deformation of the deformable portion of the needle in a range of from greater than 0% to 9%, preferably a range of from greater than 0% to 6%.
